(19) United States
(12) Patent Application Publication
(10) Pub. No.: US 2021/0098087 A1
(43) Pub. Date: Apr. 1, 2021

(54) METHOD AND SYSTEM FOR PREVENTION OF MEDICAL PROFESSIONAL BURNOUT

(71) Applicant: SE Healthcare Quality Consulting LLC, Charleston, SC (US)

(72) Inventors: Douglas G. Shand, Lancaster, PA (US); Stephanie Sargent, Charleston, SC (US)

(21) Appl. No.: 17/015,237

(22) Filed: Sep. 9, 2020

Related U.S. Application Data
(60) Provisional application No. 62/898,061, filed on Sep. 10, 2019.

(51) Int. Cl. G16H 10/20; G16H 20/00; G16H 50/30

(52) U.S. Cl. CPC G16H 10/20; G16H 50/30; G16H 20/00

(57) ABSTRACT

An iterative method for reducing medical professional burnout includes: i) conducting an initial survey of individual medical professionals in a healthcare organization; ii) analyzing the results to determine levels of medical professional burnout and organizational stress at the time of the initial survey; iii) delivering actionable results and educational tools based on the levels of medical professional burnout and organizational stress to the individual medical professionals and the healthcare organization; iv) conducting a subsequent survey at a designated time which is spaced in time from a previous survey; v) analyzing the results of the subsequent survey to determine levels of medical professional burnout and organizational stress at the time of the subsequent survey; vi) delivering actionable results and educational tools based on the levels of medical professional burnout and organizational stress determined in the subsequent survey to the individual medical professionals and the healthcare organization; and vii) sequentially repeating steps iv), v) and vi).

### FIELD OF THE INVENTION

The invention is directed to healthcare industry. In particular, the invention is directed to a system and method for the prevention of medical professional burnout, including, but not limited to physician burnout.

### BACKGROUND OF THE INVENTION

Physician and medical professional burnout is an acknowledged crisis in our healthcare system, resulting in increased medical errors and physician turnover. Medical errors are three times more likely to occur in medical units with high burnout rates, even in those ranked as “extremely safe”. This indicates that burnout may play an even more significant role as a cause of medical error than a poor safety environment.

The impact of burnout on physician and other medical professionals health and well-being has been recognized and studied over many years. Studies point out that the rate of burnout in the medical profession ranges between 40-60% depending on the specialty and is twice that of other industries in the U.S. Burned out physicians report a lack of empathy and are more likely to leave the profession. Worse yet, only a small percentage of those experiencing burnout are likely to seek help.

The impact of burnout on the physicians can be far reaching and may include: i) depersonalization, which leads to the treatment of patients as objects, not as human beings; ii) a sense of reduced personal accomplishment resulting in feelings of ineffectiveness; iii) emotional exhaustion to the point of having no desire to engage emotionally with patients; iv) a loss of meaning in the work performed; v) serious issues outside of the work environment such as alcohol abuse, deterioration of personal relationships, and even suicidal ideation; and vi) reductions in the quality of patient care.

Burnout also has an economic impact on practices, hospitals and health systems. Burnout has proven to be the highest cause of physician turnover. Lost patient-care revenue, secondary to physician turnover, is another threat, causing lulls in productivity while searches are conducted to find replacement physicians.

There is currently no method or system which obtains information from medical personnel, analyzes the information and results and uses the results to provide a program and content to help in the prevention of medical professional, including physician, burnout. There is also no system currently available which provides the healthcare organization, to which the individual medical professionals are associated, a compilation or dashboard of the results and the progress that is made in the prevention of medical professional and physician burnout for that organization.

It would be beneficial to provide an iterative method and system which gathers and analyzes results regarding medical professional burnout and uses the results to provide a program and content to help in the prevention of medical professional, including physician, burnout. It would also be beneficial to provide the healthcare organization, to which the individual medical professional, are associated, a compilation or dashboard of the results and the progress that is made in the prevention of medical professional, including physician, burnout for that organization. Properly addressing this dilemma leads to healthier and better performing medical staff, physicians and organizations and improved patient engagement and care.

### SUMMARY OF THE INVENTION

An object is to provide an iterative and progressive method and system which helps to prevent medical professional, including physician, burnout.

An object is to provide a method and system which identifies the level of medical professional burnout and organizational stress, and which delivers actionable results or recommendations and educational tools to leadership and physicians.

An object is to provide a scalable method and system for use with all medical professional practices which provides a real value for the medical professionals and their organizations.

An embodiment is directed to a method for reducing medical professional burnout, including, but not limited to physician burnout. The method includes: conducting a first survey of individual medical professionals in a healthcare organization; compiling and analyzing the results of the first survey to determine levels of medical professional burnout and organizational stress at the time of the first survey; delivering actionable results and educational tools based on the levels of medical professional burnout and organizational stress determined in the first survey to the individual medical professionals and the healthcare organization; conducting a second survey, which is different than the first survey; compiling and analyzing the results of the second survey to determine levels of medical professional burnout and organizational stress at the time of the second survey; comparing the levels of medical professional burnout and organizational stress from the first survey to the levels of medical professional burnout and organizational stress at the second survey; and delivering actionable results and educational tools based on the levels of medical professional burnout and organizational stress determined in the second survey to the individual medical professionals and the healthcare organization.

An embodiment is directed to an iterative method for reducing medical professional, including physician, burnout. The method includes: i) conducting an initial survey of individual medical professionals in a healthcare organization; ii) analyzing the results of the initial survey to determine levels of medical professional burnout and organizational stress at the time of the initial survey; iii) delivering actionable results and educational tools based on the levels of medical professional burnout and organizational stress determined in the initial survey to the individual medical professionals and the healthcare organization; iv) conducting a subsequent survey at a designated time which is spaced in time from a previous survey; v) analyzing the results of the subsequent survey to determine levels of medical professional burnout and organizational stress at the time of the subsequent survey; vi) delivering actionable results and educational tools based on the levels of medical professional burnout and organizational stress determined in the subsequent survey to the individual medical professionals and the healthcare organization; and vii) sequentially repeating steps iv), v) and vi).

Other features and advantages of the present invention will be apparent from the following more detailed description of the preferred embodiment, taken in conjunction with the accompanying drawings which illustrate, by way of example, the principles of the invention.

### BRIEF DESCRIPTION OF THE DRAWINGS

FIG. 1 is a flow chart illustrating the method for preventing medical professional, including physician, burnout according to the present invention.

### DETAILED DESCRIPTION OF THE INVENTION

The description of illustrative embodiments according to principles of the present invention is intended to be read in connection with the accompanying drawings, which are to be considered part of the entire written description. In the description of embodiments of the invention disclosed herein, the features and benefits of the invention are illustrated by reference to the preferred embodiments. Accordingly, the invention expressly should not be limited to such preferred embodiments illustrating some possible non-limiting combination of features that may exist alone or in other combinations of features, the scope of the invention being defined by the claims appended hereto.

The medical professional or physician burnout prevention method and system 10 is a comprehensive enterprise-level medical professional (including, but not limited to physician burnout tool) which can be conducted on multiple devices, including, but not limited to, computers or mobile tablets. The iterative and progressive method 10 creates the opportunity to improve dialogue between the organization and medical professionals. The medical professional burnout prevention method and system 10 includes an assessment, suggested actions and education, complete with high-quality video, audio and written materials to help the medical professional and organization actively address and prevent burnout.

Sequential surveys 12, 20 focus on organization-specific issues and new areas of stress. The program may include, but is not limited to, monthly newsletters, content for white labeling, additional video trainings and/or usage statistics to ensure continued monthly engagement and improvement. The medical professional burnout prevention system identifies the level of medical professional burnout, and in particular, but not limited to physician burnout, and organizational stress, delivering actionable results and educational tools to leadership and medical professionals.

Medical professionals will have access to tutorial content including videos, self-help tools and other instructional content. These are focused on relieving stress, building more life balance, plus improving the individual's leadership and communication skills, workflow and decision making. The medical professional burnout prevention system is built for use by all healthcare and physician practices, including large, highly organized integrated delivery systems. The medical professional burnout prevention system is scalable, generating a real value for the medical professionals, including the physicians, and their organizations.

The medical professional burnout prevention method and system 10 is sequential to ensure constant focus and improvement. For example, a survey is administered 12, the results are compiled and assessed 14, 16, a plan is developed and implemented to achieve improvements 18. A follow-up second survey 20 is then conducted at a predetermined time, for example between 9 to 12 months, driving down to specific areas identified by the initial survey and the organization. The follow up survey includes additional measurements and materials. Additional follow-up surveys 20 may also be conducted at additional predetermined times.

The assessment is weighted for impact on risk, safety and downstream consequences to the organization. In one illustrative embodiment, the results provide measures of categories such as: workplace attitudes, practice environment, work-life balance, values, respect, trust and degree of burnout. In each category, leadership receives actionable data displayed in an electronic dashboard which allows the leadership to easily view the results and track progress and improvements made based on the recommended actions.

In various embodiments, the responses of the individual medical professionals are compiled and remain anonymous. In other embodiments, the results of the individual medical professionals may be tracked for the limited purpose of providing the individual medical professionals with feedback and recommendations.

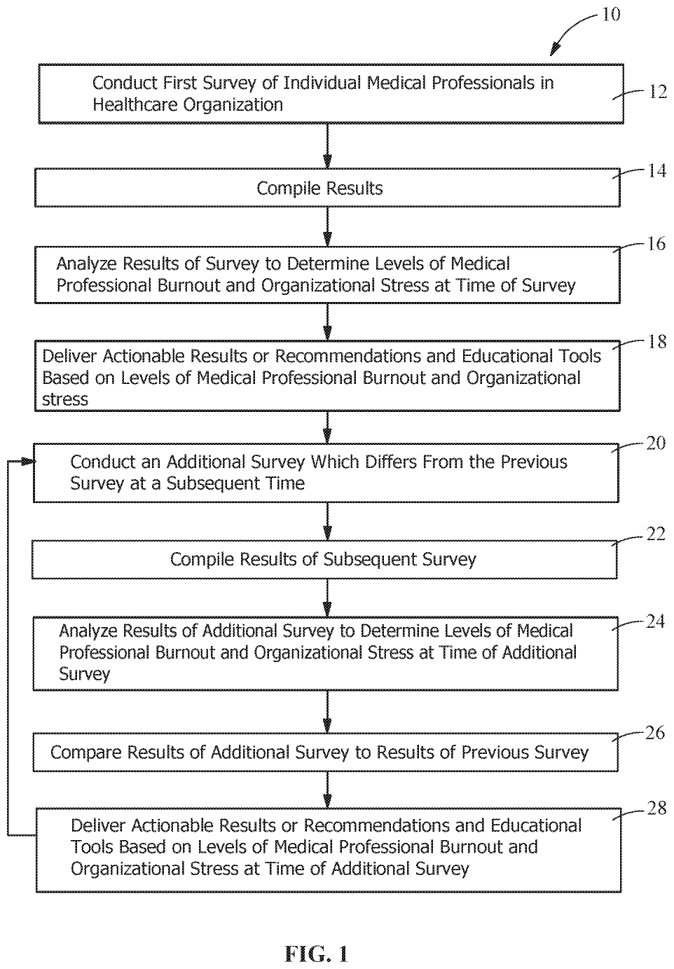

In one illustrative embodiment, the initial survey, consisting of ten questions, is given to individual medical professionals, such as physicians, in a healthcare organization, as represented by 12 in FIG. 1. The survey may consist of questions which are answered by selecting an appropriate response from a scale and other questions which require free form text entry answers, allowing the medical professional to express concerns in a narrative format.

The results of the individual medical professionals for each healthcare organization are compiled, as represented by 14, and analyzed to determine levels of medical professional burnout and organizational stress at the time of the survey, as represented by 16. Computer systems and artificial intelligence may be used to analyze the result. Based on the results, actionable recommendations may be delivered to the individual medical professionals and the healthcare organization, as represented by 18. In addition, educational tools, which are determined based on the levels of medical professional burnout and organizational stress determined in the survey, are also provided to the individual medical professionals and the healthcare organization.

After a determined period of time, one or more subsequent surveys are conducted, as represented by 20 in FIG. 1. Subsequent surveys may have a number of question which are identical to the initial survey or a previous survey. Subsequent surveys may have a number of question which are different than the initial survey or a previous survey to allow the subsequent survey to be customize for example, but not limited to, work locations to organization, roles to organization, results of previous surveys. The subsequent survey may consist of questions which are answered by selecting an appropriate response from a scale and other questions which require free form text entry answers, allowing the medical professional to express concerns in a narrative format.

The results of each of the subsequent surveys are compiled, as represented by 22, and analyzed to determine levels of medical professional burnout and organizational stress at the time of the respective subsequent survey, as represented by 24. Computer systems and artificial intelligence may be used to analyze the result. The levels of medical professional burnout and organizational stress at the time of the respective subsequent survey are compared to the levels of medical professional burnout and organizational stress from the previous survey to allow the progress of the system and method to be tracked, as represented by 26.

Based on the results of the subsequent survey, actionable results or recommendations may be delivered to the individual medical professionals and the healthcare organization. In addition, educational tools, which are determined based on the levels of medical professional burnout and organizational stress determined in the subsequent survey, are also provided to the individual medical professionals and the healthcare organization. The actionable results or recommendations and educational tools provided in response to the results of the subsequent survey may differ from the actionable results or recommendations and educational tools provided in response to the results of the previous survey.

As previously mentioned, a library of educational tools and resources is included in the medical professional burnout prevention program. The resources provide medical professionals with actionable guidance allowing organizations to take action to address and prevent burnout. Examples of the types of resources include: methodologies for recognizing and addressing burnout; instructional content to help build a better work life balance; resources to help address issues like EMR fatigue and “broken record” moments in your office day; guides and tool kits to assist employee medical professionals to navigate daily challenges; tips and resources to help leaders become more effective; and informational content to help maintain meaningful connections to patients.

After the surveys are assessed, an electronic dashboard is provided which includes organizational and work unit-level data available to leadership, as well as individualized reports for the medical professionals. The survey measures an individual's personal assessment of their level of burnout and their attitudes and perceptions about the workplace and organizational leadership. An illustrative report would include, but not be limited to: graphs and charts; links to pertinent content and tools to improve wellness; overview of overall scores; drills down to locations (such as hospital or practice locations) and work groups within hospitals or practices (where applicable); and/or filter by specialty, age, gender and location

Referring the illustrative embodiment shown in FIG. 1, the iterative method for reducing medical professional burnout 10 includes: conducting an initial or first survey of individual medical professional in a healthcare organization 12; compiling the results of the survey 14; analyzing the results of the initial or first survey to determine levels of medical professional burnout and organizational stress at the time of the initial survey 16; and delivering actionable results and educational tools based on the levels of medical professional burnout and organizational stress determined in the initial or first survey to the individual medical professionals and the healthcare organization 18. The method 10 also includes: conducting a subsequent or second survey at a designated time which is spaced in time from a previous survey 20; compiling the results of the subsequent or second survey 22; analyzing the results of the subsequent or second survey to determine levels of medical professional burnout and organizational stress at the time of the subsequent or second survey 24; comparing the results of the subsequent or second survey to the results of previous surveys 26; and delivering actionable results and educational tools based on the levels of medical professional burnout and organizational stress determined in the subsequent or second survey to the individual medical professionals and the healthcare organization 28. Steps 20, 22, 24, 26 and 28 can be repeated as desired at various or defined intervals.

By using the method and system of the present invention, medical professional burnout is reduced leading to fewer medical errors and lower malpractice exposure and expense. In addition, higher levels of medical professional, and in particular physician, trust and engagement make medical practices and organizations more profitable, stable and nimble than medical practices and organizations which do not use the method and system described herein. The system and method also leads to a more supportive culture, improved patient experience and engagement and better quality of care.

While the invention has been described with reference to a preferred embodiment, it will be understood by those skilled in the art that various changes may be made, and equivalents may be substituted for elements thereof without departing from the spirit and scope of the invention as defined in the accompanying claims. In particular, it will be clear to those skilled in the art that the present invention may be embodied in other specific forms, structures, arrangements, proportions, sizes, and with other elements, materials and components, without departing from the spirit or essential characteristics thereof. One skilled in the art will appreciate that the invention may be used with many modifications of structure, arrangement, proportions, sizes, materials and components and otherwise used in the practice of the invention, which are particularly adapted to specific environments and operative requirements without departing from the principles of the present invention. The presently disclosed embodiments are therefore to be considered in all respects as illustrative and not restrictive, the scope of the invention being defined by the appended claims, and not limited to the foregoing description or embodiments.

1. A method for reducing medical professional burnout, the method comprising:
conducting a first survey of individual medical professionals in a healthcare organization;
compiling and analyzing the results of the first survey to determine levels of medical professional burnout and organizational stress at the time of the first survey;
delivering actionable results and educational tools based on the levels of medical professional burnout and organizational stress determined in the first survey to the individual medical professionals and the healthcare organization;
conducting a second survey, which is different than the first survey;
compiling and analyzing the results of the second survey to determine levels of medical professional burnout and organizational stress at the time of the second survey;
comparing the levels of medical professional burnout and organizational stress from the first survey to the levels of medical professional burnout and organizational stress at the second survey;
delivering actionable results and educational tools based on the levels of medical professional burnout and organizational stress determined in the second survey to the individual medical professional and the healthcare organization.

2. The method for reducing medical professional burnout as recited in claim 1, further comprising:
sequentially conducting subsequent surveys at designated times which are spaced in time from a previous survey;
analyzing the results of each subsequent survey of the sequentially conducted subsequent surveys to determine levels of medical professional burnout and organizational stress at the time of the each subsequent survey;
delivering actionable results and educational tools based on the levels of medical professional burnout and organizational stress determined in the each subsequent survey to the individual medical professionals and the healthcare organization.

3. The method for reducing medical professional burnout as recited in claim 1, further comprising:
providing a report which includes organizational data to leadership of the healthcare organization.

4. The method for reducing medical professional burnout as recited in claim 1, further comprising:
providing a report which includes an individual medical professional's data to the individual medical professional.

5. The method for reducing medical professional burnout as recited in in claim 1, wherein the educational tools include videos.

6. The method as recited in claim 1, wherein the educational tools include self-help tools.

7. The method for reducing medical professional burnout as recited in claim 1, wherein the second survey has questions which are customized according to the results of the first survey.

8. The method for reducing medical professional burnout as recited in claim 1, wherein the second survey has questions which are customized according the instructions of the healthcare organization.

9. An iterative method for reducing medical professional burnout, the method comprising:
i) conducting an initial survey of individual medical professionals in a healthcare organization;
ii) analyzing the results of the initial survey to determine levels of medical professional burnout and organizational stress at the time of the initial survey;
iii) delivering actionable results and educational tools based on the levels of medical professional burnout and organizational stress determined in the initial survey to the individual medical professionals and the healthcare organization;
iv) conducting a subsequent survey at a designated time which is spaced in time from a previous survey;
v) analyzing the results of the subsequent survey to determine levels of medical professional burnout and organizational stress at the time of the subsequent survey;
vi) delivering actionable results and educational tools based on the levels of medical professional burnout and organizational stress determined in the subsequent survey to the individual medical professionals and the healthcare organization;
vii) sequentially repeating steps iv), v) and vi).

10. The iterative method for reducing medical professional burnout as recited in claim 9, further comprising:
comparing the levels of medical professional burnout and organizational stress from the subsequent survey to the levels of medical professional burnout and organizational stress at the previous survey.

11. The iterative method for reducing medical professional burnout as recited in claim 10, further comprising:
providing respective reports for the initial survey and each subsequent survey which includes organizational data to leadership of the healthcare organization.

12. The iterative method for reducing medical professional burnout as recited in claim 10, further comprising:
providing respective reports for the initial survey and each subsequent survey report which includes an individual medical professional's data to the individual medical professional.

13. The iterative method for reducing medical professional burnout as recited in claim 9, wherein the educational tools include videos.

14. The iterative method for reducing medical professional burnout as recited in claim 9, wherein the educational tools include self-help tools.

15. The iterative method for reducing medical professional burnout as recited in claim 9, wherein each subsequent survey has questions which are customized according to the results of the previous surveys.

16. The iterative method for reducing medical professional burnout as recited in claim 9, wherein each subsequent survey has questions which are customized according the instructions of the healthcare organization.
